(19) United States
(12) Patent Application Publication
(10) Pub. No.: US 2006/0275779 A1
(43) Pub. Date: Dec. 7, 2006

(54) Method and apparatus for molecular analysis using nanowires

(76) Inventors: Zhiyong Li, Palo Alto, CA (US); Shih-Yuan Wang, Palo Alto, CA (US); Wei Wu, Mountain View, CA (US); M. Saif Islam, Sacramento, CA (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/144,588

(22) Filed: Jun. 3, 2005

(51) Int. Cl. C12Q 1/68; C12M 1/34

(52) U.S. Cl. 435/6; 435/287.2; 977/924

(57) ABSTRACT

Devices and methods for detecting the constituent parts of biological polymers are disclosed. A molecular analysis device comprises a molecule sensor and a molecule guide. The molecule sensor comprises a nanowire operably coupling a first terminal and a second terminal and a nitrogenous material disposed on the nanowire. The nitrogenous material is configured to interact with an identifiable configuration of a molecule such that the molecule sensor develops a conductance change responsive to the interaction. The molecule guide is configured for guiding at least a portion of the molecule near the molecule sensor to enable the interaction.

### FIELD OF THE INVENTION

The present invention relates to chemical analysis using nanoelectronic circuits. More particularly, the present invention relates to systems for determining chemical sequences of biological polymers using nanoscale transport systems and nanowires.

### BACKGROUND OF THE INVENTION

Determining the sequence of a Deoxyribonucleic acid (DNA) molecule is, conventionally, a difficult and expensive chemical process. However, with the rapid growth in nanotechnology new methods may be devised to increase accuracy, speed, and cost of determining the constituent parts of biological polymer such as proteins, DNA, and ribonucleic acid (RNA).

Various methods have been developed for determining the chemical composition of portions of a DNA strand or the chemical composition of an entire DNA strand. One such method involves creating a micro-array with hundreds or thousands of patches of single stranded DNA, which are often referred to as probes, attached to various locations on a substrate such as glass or silicon.

When using this DNA detection method, the DNA to be examined is first transcribed into RNA. RNA is a chemical very similar to DNA that can encode the same information as DNA. The RNA can then be used to create single stranded DNA (ssDNA) copies of the RNA. Fluorescent molecules, also referred to as tags, are then bonded onto the new single stranded DNA molecules.

When these tagged single stranded DNA molecules are washed over the micro-array, they will bond and stick to any of the single stranded DNA probes having a gene sequence with bases that are complementary to, but arranged in the same order as, the bases of the tags. Then, a light source exposing the micro-array causes the tagged DNA molecules that have stuck to the micro-array to fluoresce. The fluorescent glow can be detected and, based on where the various DNA tags were placed and their corresponding sequence, the sequence of the portion of the DNA stuck to that site can be determined.

Unfortunately, this process requires a significant number of chemical and optical steps to determine various portion of a DNA sequence. In addition, the detection is limited to the variety of DNA probes on the micro-array. Long probes, with a large number of sequences can detect a significant match, but it becomes difficult to place every possible variation of long probes on a single micro-array. On the other hand, short probes may be incapable of detecting a desired long sequence.

Another proposed detection method involves examining a polymerase chain reaction replication process. An RNA polymerase may attach to a DNA molecule and begin separating the DNA strand. The RNA polymerase then traverses along the DNA strand opening newer regions of the DNA strand and synthesizing an RNA strand matching the opened portions of the DNA. As the RNA polymerase traverses along the DNA, the portion of the DNA opened by the RNA polymerase closes down and re-bonds after leaving the RNA polymerase. In this detection method, the RNA polymerase is attached to an electronic device, such as a single electron transistor. Whenever the polymerase replication takes place, a charge variation may occur on the single electron transistor for each portion of the DNA molecule opened up by the RNA polymerase. By detecting these charge variations, the composition of the portion of the DNA molecule that is transcribed can be determined.

Unfortunately, the polymerase chain reaction method relies on the occurrence of this biological process of replication. In addition, the RNA polymerase replication only begins and ends at certain defined points of the DNA strand. As a result, it may be difficult to discover all portions of the DNA strand to be examined.

A device and method with the flexibility to examine the entire sequence of a DNA strand, without requiring complicated chemical and optical processing is needed. A molecule detection system using nanoelectronic devices without the requirement of a biological replication process may be a smaller and less costly system than conventional approaches. This integrated molecule detection system should be easier to use and be adaptable to detection of a variety of predetermined sets of bases within DNA molecules. Furthermore, this molecule detection system should be integrated with other electronic devices for further analysis and categorization of the detected molecules.

### BRIEF SUMMARY OF THE INVENTION

The present invention, in a number of embodiments, includes molecular analysis devices and methods for detecting the constituent parts of biological polymers. An exemplary embodiment of a molecular analysis device comprises a molecule sensor and a molecule guide. The molecule sensor comprises a nanowire operably coupling a first terminal and a second terminal and a nitrogenous material disposed on the nanowire. The nitrogenous material is configured to interact with an identifiable configuration of a molecule such that the molecule sensor develops a conductance change responsive to the interaction. The molecule guide is configured for guiding at least a portion of the molecule substantially near the molecule sensor to enable the interaction.

Another exemplary embodiment of a molecular analysis device comprises a plurality of molecule sensors and a molecule guide. Each molecule sensor of the plurality includes a nanowire operably coupling a first terminal and a second terminal and a nitrogenous material disposed on the nanowire. The nitrogenous material is configured to interact with an identifiable configuration of a molecule such that the molecule sensor develops a conductance change responsive to the interaction. The molecule guide is configured for guiding at least a portion of the molecule substantially near the nitrogenous material of each molecule sensor of the plurality to enable the interaction.

Another exemplary embodiment includes a method of detecting a molecule. The method includes guiding at least a portion of the molecule near a molecule sensor, which includes a nanowire disposed in a molecule guide. The method further includes interacting an identifiable configuration of the molecule and a nitrogenous material disposed on the nanowire. The method also includes sensing a conductance change in the molecule sensor responsive to the interaction.

### BRIEF DESCRIPTION OF THE SEVERAL VIEWS OF THE DRAWINGS

While the specification concludes with claims particularly pointing out and distinctly claiming that which is regarded as the present invention, the advantages of this invention can be more readily ascertained from the following description of the invention when read in conjunction with the accompanying drawings in which:

FIG. 1A is a three dimensional view of a portion of a DNA molecule;

FIG. 1B is a flat view of a portion of a DNA molecule showing various possible base pair bondings;

FIG. 2A is a top view of an exemplary molecular analysis device including a nanochannel and one or more molecule sensors disposed in the nanochannel and near the nanochannel;

FIG. 2B is a top view of an exemplary molecular analysis device including a plurality of nanochannels and molecule sensors disposed in the nanochannel;

FIGS. 3A, 3B, 3C, and 3D are three dimensional views of exemplary configurations of nanochannels useful in practicing the present invention;

FIG. 4A is a top view of an exemplary molecular analysis device including a nanopore and one or more molecule sensors;

FIG. 4B is a top view of an exemplary molecular analysis device including a plurality of nanopores and a plurality of molecule sensors;

FIG. 4C is a three dimensional view of an exemplary configuration of a nanopore and a molecule sensor;

FIG. 5 is a top view of a plurality of exemplary nanowires in a nanochannel and a nucleic acid chain;

FIG. 6 is a top view of an exemplary nanowire including a nitrogenous material disposed on the nanowire and an exemplary bonding to a nucleic acid chain;

FIG. 7 is a top view of an exemplary nanowire including an oligonucleotide disposed on the nanowire and an exemplary bonding to a nucleic acid chain;

FIGS. 8A is a graphical view illustrating a lack of a conductance change in a nanowire with no bonding event;

FIGS. 8B is a graphical view illustrating a conductance increase in a p-type nanowire when a bonding event occurs;

FIGS. 8C is a graphical view illustrating a conductance decrease in an n-type nanowire when a bonding event occurs; and

FIG. 9 is a top view of an exemplary embodiment of a molecular analysis device including a large number of molecule sensors configured to detect a variety of molecule configurations.

### DETAILED DESCRIPTION OF THE INVENTION

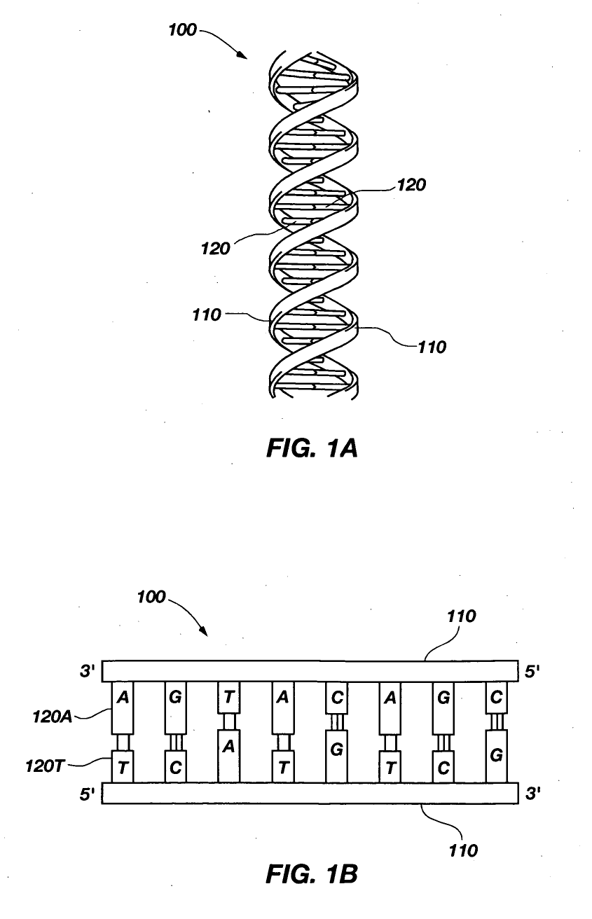

The present invention, in a number of embodiments, includes structures, devices, and methods for use in detecting the molecular structure of biological polymers. As illustrated in FIGS. 1A and 1B, an example of one such biological polymer is Deoxyribonucleic acid (DNA). A DNA molecule 100 comprises a double helix structure including two backbone strands 110 on the outside of the double helix. The backbone strands 110 are a structure made up of sugar-phosphate polymer strands. Between the two backbone strands 110 are pairs of bases 120 configured similar to ladder rungs. The bases 120 connecting the strands consist of four types: adenine 120A (A), thymine 120T (T), guanine 120G (G), and cytosine 120C (C). RNA, which is closely related to DNA, comprises a similar structure including the A, G, and C bases of DNA. However, in RNA, rather than bonding with T, A bonds with the molecule uracil (U) (not shown), which is closely related to T. In addition, while RNA can form a double helix, in nature it generally exists as a single strand.

Each of the base molecules 120 comprise nitrogenous compounds in various configurations. The base molecules 120 may bond with each other to form base pairs. As shown in FIG. 1B, T may form two weak hydrogen bonds with A, while C may form three weak hydrogen bonds with G. These weak bonds between the base pairs allow a DNA strand to be separated into two complementary single stranded molecules. A single human DNA molecule may include as many as three billion of these base pairs.

Another way of characterizing the constituent parts of a DNA strand is to consider the various bases 120 chemically bonded to a sugar. In this form, the resultant molecule is often referred to as a nucleoside. Each nucleoside includes a sugar molecule bonded to one of the various bases 120. A nucleoside with a phosphate molecule bonded to the sugar portion of the nucleoside is often referred to as a nucleotide. Thus, each strand of a DNA molecule may be considered as a plurality of nucleotides bonded together, wherein the bonds form at the sugar-phosphate portion of each nucleotide to form the backbone 110 of the strand. Nucleotides join together to form the backbone strands 110 by a 5′-3′ phosphodiester linkage, giving the strands a directionality. Thus, the 5′ end of the strand has a free phosphate group and the 3′ end has a free hydroxyl group. In double stranded DNA, the backbone strands 110 run in opposite directions such that each end of the double strand has a 5′ end on one backbone strand 110 and a 3′ end on the other backbone strand 110.

A section of single stranded DNA including a small plurality of nucleotides is often referred to as an oligonucleotide. These oligonucleotides are conventionally used as the tags in the prior art DNA micro-arrays previously described.

In genetic coding, an oligonucleotide comprising three consecutive nucleotides along RNA or single stranded DNA is often referred to as a codon. Any three consecutive nucleotides of A, C, G, and T (or U for RNA), can be combined in 64 (i.e., 4³) possible combinations. The 20 different amino acids are specified by these 64 different codons and are represented by more than one codon. For example, the amino acid Alanine may be represented by the codons GCA, GCC, GCG, and GCU.

Polypeptides and proteins (one or more polypeptide chains) are composed of a linear chain of amino acids covalently linked by peptide bonds. In addition to the codons that specify the various amino acids, some codons are defined as start codons and stop codons. These start and stop codons define the beginning and ending of the sequence of amino acids to be formed that ultimately form any given polypeptide or protein. Thus, identification of the various amino acids by direct identification of the 64 possible codons is possible.

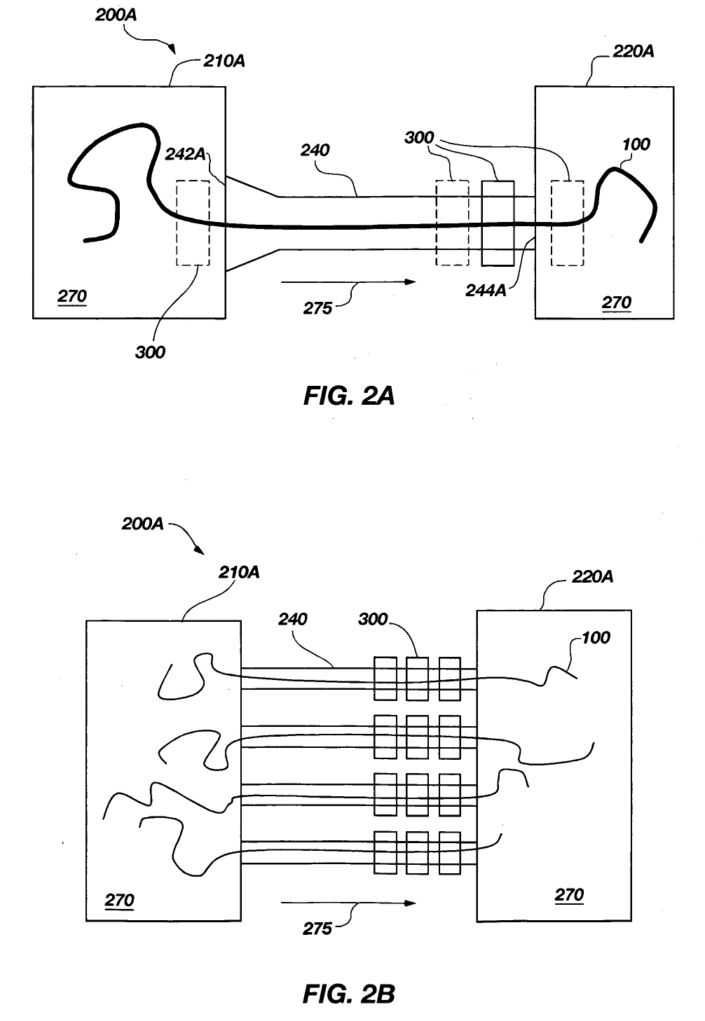

FIG. 2A illustrates an exemplary embodiment of a molecular analysis device 200A for analyzing biological polymers such as nucleic acid chains, including DNA and RNA. The molecular analysis device 200A includes a supply reservoir 210A, an accumulation reservoir 220A, a molecule guide (such as a nanochannel 240 shown in FIG. 2A), and at least one molecule sensor 300. In addition, a transport medium 270, such as, for example, an electrolyte solution, may be contained within the supply reservoir 210A, the nanochannel 240, and the accumulation reservoir 220A. At least one nucleic acid chain 100 may be disposed within the transport medium 270. The molecule sensor 300 is described in more detail below.

The nanochannel 240 may be configured as a nanofluidic channel for carrying the nucleic acid chain 100 in the transport medium 270 from the supply reservoir 210A, through the nanochannel 240, to the accumulation reservoir 220A in the transport direction 275 shown. Alternatively, the transport medium 270 may be configured for carrying the nucleic acid chain 100 from the accumulation reservoir 220A, through the nanochannel 240 to the supply reservoir 210A. Various methods may be used to transport the nucleic acid chain 100 through the nanochannel 240, such as, by way of example, electrokinetic flow, electroosmotic flow, hydrostatic pressure, hydrodynamic pressure, and hydromagnetic flow. These transport mechanisms may be caused by mechanical, magnetic, electrical field, heat-induced, and other methods known to a person of ordinary skill in the art.

Electrophoresis causes the movement of particles that are suspended in a medium to which an electromotive force is applied. Particularly, a particle or molecule having an electrical charge will experience an electromotive force when positioned within an electrical field. Nucleic acid chains 100 are good candidates for electrophoresis because they carry multiple negative charges due to the phosphate group and the phosphodiester backbone strand 110 (FIGS. 1A and 1B). Thus, when electrodes (not shown) with a voltage differential are placed in the transport medium 270, the nucleic acid chains 100 will migrate toward the more positive electrode. By way of example, if an electrode with a ground potential is placed in the supply reservoir 210A and an electrode with a positive voltage is placed in the accumulation reservoir 220A, nucleic acid chains 100 in the transport medium 270 will migrate from the supply reservoir 210A, through the nanochannel 240, and toward the electrode in the accumulation reservoir 220A. Furthermore, the movement rate or velocity of the nucleic acid chain 100 substantially correlates with the voltage bias between the electrodes. As a result, a first approximation of the nucleic acid chain 100 velocity may be determined, which may be used by, and refined by, signal processing analysis in combination with signal data from the molecule sensor 300 to determine the constituent parts of the nucleic acid chain 100.

Other transport mechanisms may rely on nanofluidic flow of the transport medium 270 itself, with the nucleic acid chain 100 being carried along with the transport medium 270. For example, electrokinetic flow (often referred to as electroosmotic flow) is generated in a similar manner to electrophoresis by electrodes (not shown) in the supply reservoir 210A and the accumulation reservoir 220A. Electrokinetic flow of the transport medium 270 may generally require higher voltage potentials to cause transport medium 270 flow than the voltage required to cause electrophoretic movement of the nucleic acid chains 100. Thus, nucleic acid chain 100 movement may be substantially electrophoretic or may be a combination of electrophoretic movement and movement caused by electrokinetic flow of the transport medium 270.

Yet another transport mechanism may rely on pressure driven flow. In very small channels, such as nanochannels 240, a small pressure differential may be developed by applying a temperature differential between the supply reservoir 210A and the accumulation reservoir 220A. This small pressure differential may cause the flow of the transport medium 270, and nucleic acid chains 100 within the transport medium 270, from one reservoir (210A, 220A) to the other reservoir (220A, 210A).

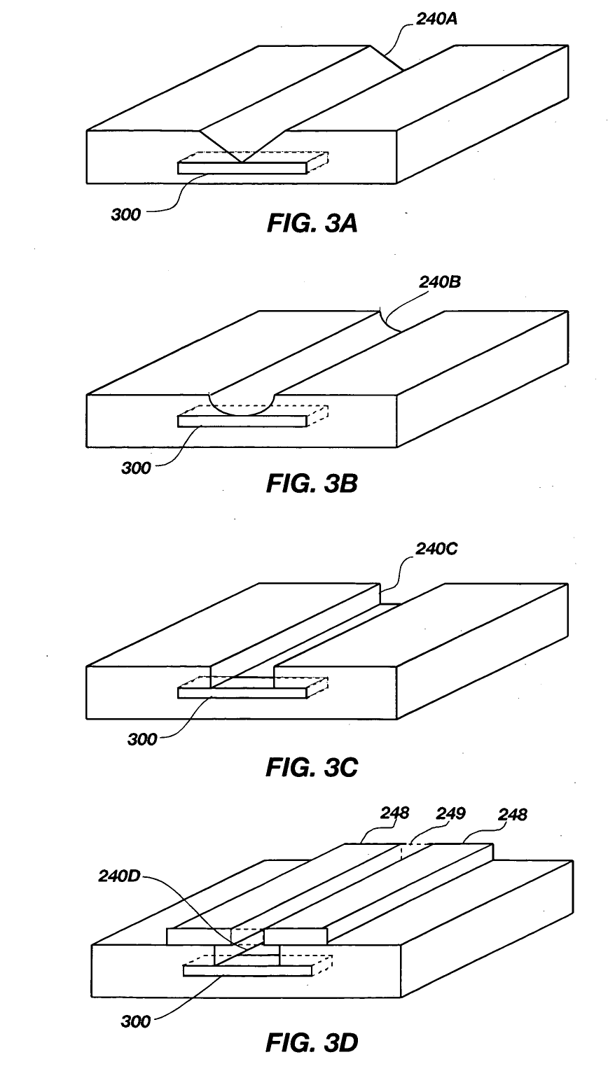

As shown in FIGS. 3A through 3D, the nanochannel 240 may be formed in a variety of configurations and cross sections. FIG. 3A illustrates a nanochannel 240A with a triangular cross section and a molecule sensor 300 positioned in the nanochannel 240A. FIG. 3B illustrates a nanochannel 240B with a semi-elliptical cross section and a molecule sensor 300 positioned in the nanochannel 240B. FIG. 3C illustrates a nanochannel 240C with a rectangular cross section and a molecule sensor 300 positioned in the nanochannel 240C. FIG. 3D illustrates a nanochannel 240D with a rectangular cross section and a molecule sensor 300 positioned in the nanochannel 240D. FIG. 3D further illustrates a partial channel cover 248 formed over a portion of the nanochannel 240 so that the nanochannel 240 is partially enclosed. Alternatively, a full channel cover 249 may be formed over the entire nanochannel 240 as shown by the dashed lines indicating a fully enclosed nanochannel 240.

Other nanochannel 240 cross sections are contemplated as being within the scope of the present invention, such as, by way of example and not limitation, circular, semi-circular, triangular, square, and hexagonal. Of course, the partially enclosed and fully enclosed nanochannel embodiments shown in FIG. 3D may be used with any of the various cross sections.

The nanochannels 240, partial channel covers 248, and full channel covers 249 may be fabricated using a variety of lithographic techniques, nano-imprint lithographic techniques, self-assembly techniques, template synthesis, wafer bonding, or combinations thereof. Additionally, the nanochannel 240 may be formed initially as a fully enclosed structure without the need for additional steps to form a partial channel cover 248 or full channel cover 249.

The length of the nanochannel 240 may vary from nanometers to orders of magnitude longer for adaptation to various applications and nucleic acid chain 100 lengths to be analyzed. Furthermore, the nanochannels 240 may include curves of a radius favorable to nucleic acid chain 100 flow and may be configured to enable long channels in a restricted area.

The nanochannel 240 is configured to at least partially straighten the nucleic acid chain 100 such that loops do not form within the channel and such that the nucleic acid chain 100 may be presented substantially near the molecule sensor 300. To ensure that loops do not form within the channel, in a particular embodiment, the channel cross section may need to be about twice the persistence length of the nucleic acid chain 100, or less. At room temperature, the persistence length for double stranded DNA is about 50 nm (i.e., L). Therefore, the nanochannel 240 should be about 100 nm (i.e., 2 L) or less to ensure that loops do not form.

To ensure that the nucleic acid chain 100 is presented substantially near the molecule sensor 300, the nanochannel 240 may need to be significantly narrower than the width needed to keep the nucleic acid chain 100 from forming loops. Thus, nanochannel 240 cross section dimensions may vary depending on the type of molecule sensor 300 used, as explained more fully below in the discussion of the molecule sensor 300. Furthermore, the cross section dimensions may vary along the length of the nanochannel 240. For example, a nanochannel 240 may have a relatively wide cross section for much of its length and narrow down to a smaller cross section near a molecule sensor 300.

Returning to FIG. 2A, a molecule sensor 300 is shown in the nanochannel 240 near an exit point 244A of the nanochannel 240. Other optional molecule sensors 300 are also shown to illustrate the flexibility and possibilities for positioning of the molecule sensors 300 relative to the nanochannel 240 and nucleic acid chain 100. It may be desirable to place multiple molecule sensors 300 in various positions to detect various portions of the nucleic acid chain 100. For example, an optional molecule sensor 300 is shown in the nanochannel 240, an optional molecule sensor 300 is shown in the supply reservoir 210A substantially near an entrance point 242A of the nanochannel 240, and an optional molecule sensor 300 is shown in the accumulation reservoir 220A substantially near the exit point 244A of the nanochannel 240. Molecule sensors 300 outside of the nanochannel 240 (i.e., near the entrance point 242A or exit point 244A) may be placed in a location where the nucleic acid chain 100 is still presented substantially near the molecule sensors 300 and where the nucleic acid chain 100 has not assumed an un-straightened configuration. It will be understood by those of ordinary skill in the art that the labeling of entrance point 242A and exit point 244A are arbitrary, as the molecular analysis device 200A may be configured to cause flow of the nucleic acid chain 100 in either direction through the nanochannel 240.

FIG. 2B illustrates a plurality of nanochannels 240 all coupled to a single supply reservoir 210A and a single accumulation reservoir 220A, with a nucleic acid chain 100 in each of the plurality of nanochannel 240. In addition, each of the nanochannels 240 is shown with a plurality of molecule sensors 300 in the nanochannels 240 and a transport direction 275 from the supply reservoir 210A to the accumulation reservoir 220A. A person of ordinary skill in the art will appreciate that many configurations of reservoirs (210A, 220A), nanochannels 240, and molecule sensors 300 are contemplated within the scope of the invention.

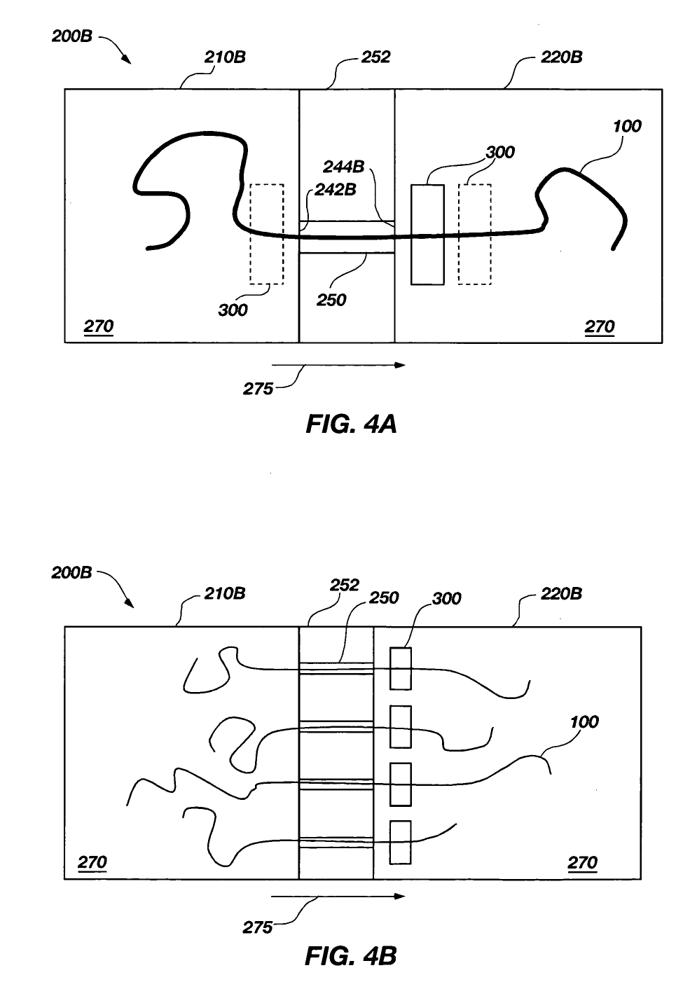

FIG. 4A illustrates another exemplary embodiment of a molecular analysis device 200B for analyzing biological polymers. The molecular analysis device 200B includes a supply reservoir 210B, an accumulation reservoir 220B, a molecule guide (also referred to as a nanopore 250 in the embodiment of FIG. 4A), and a molecule sensor 300. In addition, a transport medium 270, such as, for example, an electrolyte solution, may be contained within the supply reservoir 210B, the nanopore 250, and the accumulation reservoir 220B. At least one nucleic acid chain 100 may be disposed within the transport medium 270. The molecule sensor 300 is described in more detail below.

The nanopore 250 may be configured for carrying the nucleic acid chain 100 in the transport medium 270 from the supply reservoir 210B, through the nanopore 250, to the accumulation reservoir 220B in the transport direction 275 shown. Alternatively, the transport medium 270 may be configured for carrying the nucleic acid chain 100 from the accumulation reservoir 220B, through the nanopore 250, to the supply reservoir 210B. The same methods discussed above for transportation of the nucleic acid chain 100 through the nanochannel 240 of FIGS. 1 and 2 are applicable for transportation of the nucleic acid chain 100 through the nanopore 250.

A nanopore 250, as shown in FIGS. 4A, 4B, and 4C has an opening of from about 1 nanometer to about 100 nanometers, in a membrane 252. The membrane 252 may comprise an organic or inorganic material, which may be fabricated using a variety of lithographic techniques, nano-imprint lithographic techniques, self-assembly techniques, template synthesis, wafer bonding, or combinations thereof.

The nanopore 250 may be cylindrical in shape (as shown in FIG. 4C) or may include other cross sectional shapes such as, by way of example only, triangular, square, hexagonal, and octagonal. The figures illustrating nanopores 250 in membranes 252 are generally shown with a nanopore 250 configured horizontally through a vertical membrane 252. However, the membrane 252 may be disposed horizontally, with a vertical nanopore 250 therethrough, or any other suitable configuration, so long as the nanopore 250 may be configured to present successive segments of the nucleic acid chain 100 substantially near the molecule sensor 300, as explained below.

In a particular embodiment, the nanopore 250 may be about 100 nm or less to ensure the nucleic acid chain 100 does not pass through the nanopore 250 in some type of looped configuration, as explained above in the discussion of persistence length. To ensure that the nucleic acid chain 100 is presented substantially near the molecule sensor 300, the nanopore 250 may need to be significantly narrower than the width needed to keep the nucleic acid chain 100 from forming loops. Thus, nanochannel 240 cross section dimensions may vary depending on the type of molecule sensor 300 used, as explained more fully below in the discussion of the molecule sensor 300.

The membrane 252 may be a wide variety of thicknesses because the invention uses the nanopore 250 as a presentation and transport mechanism, rather than a sensing mechanism. A relatively thin membrane 252 may enable more uniform nanopores 250. A relatively thick membrane 252 may assist in straightening the nucleic acid chain 100 in the vicinities of the nanopore 250 entrance point 242B and nanopore 250 exit point 244B, allowing additional molecule sensors 300 to be lined up in the area where the nucleic acid chain 100 remains relatively straight such that it can be transported substantially close to a plurality of molecule sensors 300.

In FIG. 4A, a molecule sensor 300 is shown substantially near an exit point 244B of the nanopore 250. Other optional molecule sensors 300 are also shown to illustrate the flexibility and possibilities for positioning of the molecule sensors 300 relative to the nanopore 250 and nucleic acid chain 100. It may be desirable to place multiple molecule sensors 300 in positions to detect various portions of the nucleic acid chain 100. As examples, an optional molecule sensor 300 is shown in the supply reservoir 210B substantially near an entrance point 242B of the nanopore 250, and an additional molecule sensor 300 is shown in the accumulation reservoir 220B near the exit point 244B of the nanopore 250. Molecule sensors 300 near the entrance point 242B or exit point 244B may be placed in a location where the nucleic acid chain 100 is presented substantially near the molecule and wherein the nucleic acid chain 100 has not assumed its folded (un-straightened) configuration. It will be understood by those of ordinary skill in the art that the labeling of entrance point 242B and exit point 244B are arbitrary, as the molecular analysis device 200 may be configured to cause flow of the nucleic acid chain 100 in either direction through the nanopore 250.

FIG. 4B illustrates a plurality of nanopores 250 all coupled to a single supply reservoir 210A and a single accumulation reservoir 220B, with a nucleic acid chain 100 in each of the plurality of nanopores 250 and a transport direction 275 from the supply reservoir 210B to the accumulation reservoir 220B. A person of ordinary skill in the art will appreciate that many configurations of reservoirs (210B, 220B), nanopores 250, and molecule sensors 300 are contemplated within the scope of the invention.

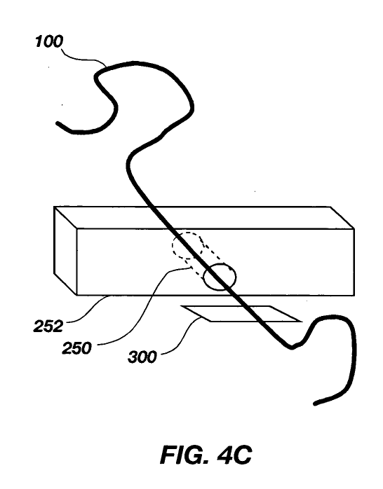

FIG. 5 illustrates three exemplary molecule sensors configured as nanowires 330. Each nanowire 330 is disposed on a substrate (not shown) between a first terminal 310 and a second terminal 320. These terminals (310, 320) may be used to couple to an apparatus for sensing a conductance change, couple to other semiconductor circuitry on the substrate for sensing a conductance change in the nanowire 330, or combinations thereof, as explained below. The exemplary nanowires 330 of the FIG. 5 embodiment are shown disposed in a nanochannel 240 with a nucleic acid chain 100 disposed in the nanochannel 240 and crossing the nanowires 330.

The exemplary nanowires 330 may be fabricated as silicon nanowires 330 on a silicon substrate with an insulating silicon dioxide layer, as an example. However, other substrates suitable for bearing and fabricating semiconductive nanowires 330 are contemplated as being within the scope of the present invention. In addition, the exemplary nanowires 330 may be doped by ion implantation using a doping material, such as, for example, boron and phosphorous to create p-type doping and an n-type doping, respectively. A p-doped nanowire 330P and an n-doped nanowire 330N are illustrated in FIG. 5

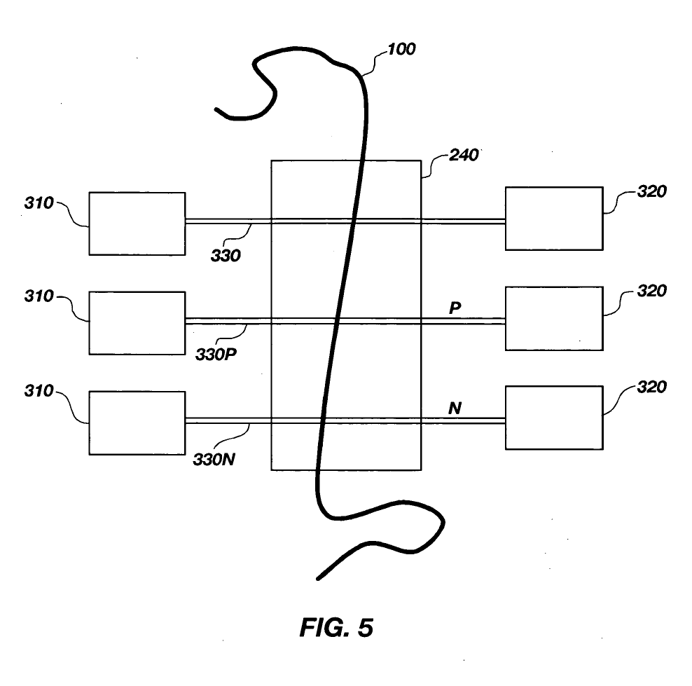

FIG. 6 illustrates an exemplary molecule sensor including a nitrogenous material 350 disposed on the nanowire 330. For example, for detecting portions of a nucleic acid chain 100, the nitrogenous material 350 may comprise a base 120 selected from the group consisting of adenine 120A, thymine 120T, uracil 120U, cytosine 120C, and guanine 120G. Furthermore, the nitrogenous material 350 on the nanowire 330 may also include a sugar bonded to the base 120 or a sugar-phosphate bonded to the base 120. By way of example, FIG. 6 illustrates the nitrogenous material 350 guanine 120G. Guanine 120G is illustrated in FIG. 6 as a symbol to show functional interaction with the nucleic acid chain 100. However, generally, the entire nanowire 330 may be coated with the nitrogenous material 350.

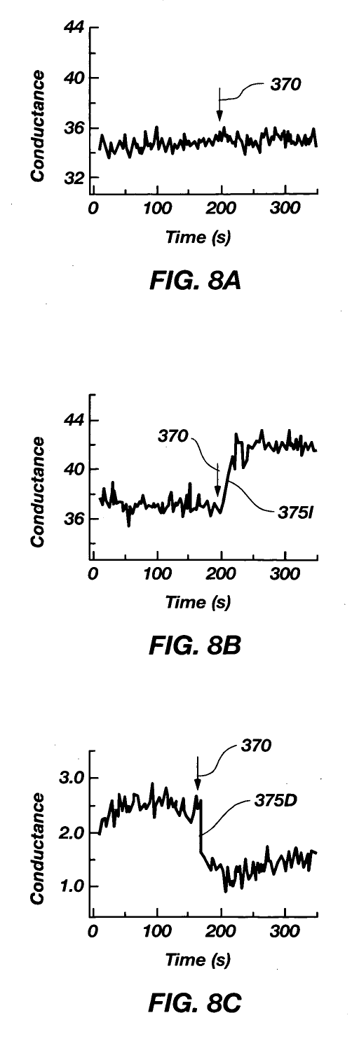

As the nucleic acid chain 100 passes substantially near the coated nanowire 330, a base (in this example, C) of the nucleic acid chain 100 that is complementary to the nitrogenous material 350 (in this example, G) on the nanowire 330 may react with the nitrogenous material 350. This reaction may take the form of a transitory chemical bond between the complementary base on the nucleic acid chain 100 and the nitrogenous material 350 on the nanowire 330. The transitory chemical bond may cause a conductance change 375 (shown in FIGS. 8B and 8C) in the nanowire 330.

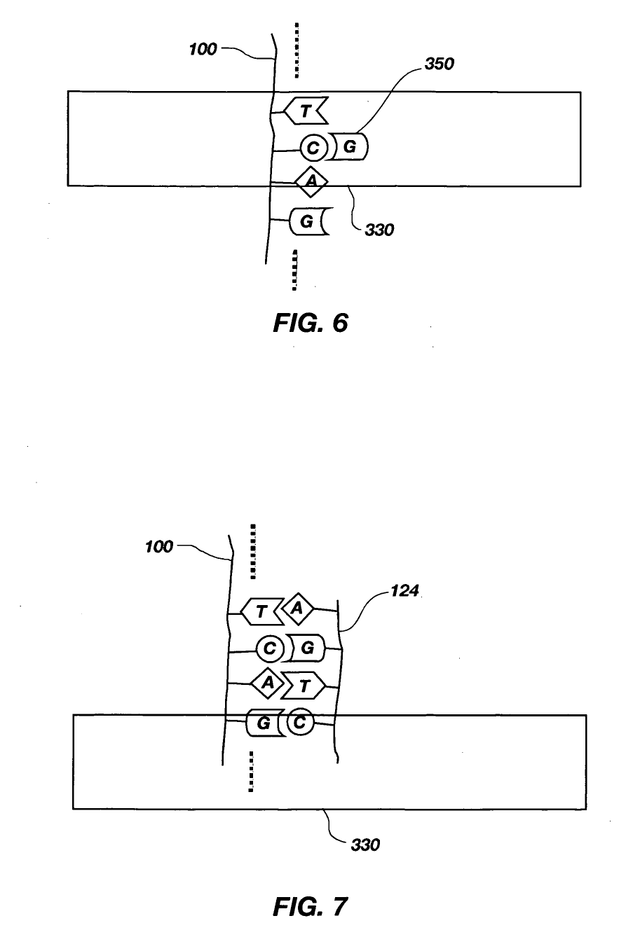

FIG. 7 illustrates another exemplary molecule sensor including a nitrogenous material comprising an oligonucleotide 124 attached to the nanowire 330. The oligonucleotide 124 may include many combinations of nucleotides and may be of various lengths to comprise a specific combination of nucleotides that may be of interest. By way of example, FIG. 7 illustrates an oligonucleotide 124 including four nucleotides in the series of C, T, G, and A.

The attachment of the oligonucleotide 124 to the nanowire 330 may be accomplished with a variety of methods known to those of ordinary skill in the art, such as, by way of example only, the methods used in micro-arrays using fluorescent tags.

As the nucleic acid chain 100 passes near the attached oligonucleotide 124, if a complementary sequence of bases passes near the attached oligonucleotide 124, a transitory chemical bond (i.e., hybridization) may occur between the oligonucleotide 124 and the complementary sequence on the nucleic acid chain 100. In the FIG. 7 exemplary embodiment, the oligonucleotide 124 comprising the sequence C, T, G, A, may hybridize with the complementary sequence G, A, C, T on the nucleic acid chain 100. As with the single base example of FIG. 6, this transitory chemical bond between the nucleic acid chain 100 and the attached oligonucleotide 124, will cause a conductance change 375 (shown in FIGS. 8B and 8C) of the nanowire 330. A plurality of molecule sensors 300 configured with a variety of oligonucleotides 124 may be useful in determining different specific characteristics of any given nucleic acid chain 100.

The transitory chemical bond results from weak hydrogen bonds between the base 120 (or oligonucleotide 124) on the nanowire 330, and the nucleic acid chain 100. The transitory chemical bond may be broken, allowing continued transportation of the nucleic acid chain 100, by the motive force causing transportation of the nucleic acid chain 100, thermal energy, optical energy, or combinations thereof.

FIGS. 8A, 8B, and 8C illustrate measurement of conductance characteristics of the nanowires 330, 330P and 330N previously described with reference to FIG. 5. FIG. 8A illustrates conductance of a p-doped nanowire 330P and an oligonucleotide 124 attached to the p-doped nanowire 330P. An introduction point 370 indicates the point in time where a nucleic acid chain 100 with a non-complementary sequence approaches substantially near the oligonucleotide 124. As can be seen in FIG. 8A, there is not a substantial difference in the conductance of the p-doped nanowire 330P.

FIG. 8B illustrates conductance of a p-doped nanowire 330P and an oligonucleotide 124 attached to the p-doped nanowire 330P. An introduction point 370 indicates the point in time where a nucleic acid chain 100 with a complementary sequence approaches substantially near the oligonucleotide 124. When the nucleic acid chain 100 bonds with the base 120 (or oligonucleotide 124) on the p-doped nanowire 330P, the increase of negative charge introduced by the nucleic acid chain 100 enhances the carrier concentration in the p-doped nanowire 330P, resulting in a measurable increase 375I in the conductance of the p-doped nanowire 330P.

FIG. 8C illustrates conductance of an n-doped nanowire 330N and an oligonucleotide 124 attached to the n-doped nanowire 330N. An introduction point 370 indicates the point in time where a nucleic acid chain 100 with a complementary sequence approaches substantially near the oligonucleotide 124. When the nucleic acid chain 100 bonds with the base 120 (or oligonucleotide 124) on the n-doped nanowire 330N, the increase of negative charge introduced by the nucleic acid chain 100 reduces the carrier concentration in the n-doped nanowire 330N, resulting in a measurable decrease 375D in the conductance of the n-doped nanowire 330N.

Additional electronics may be provided on the substrate, as additional semiconductor devices may be used to sense the conductance change. Also, signal processing hardware (on the substrate or external to the substrate), signal processing software, or a combination thereof, may then be used to gather and process data related to the times when complimentary bases 120 (or complimentary oligonucleotides 124) are substantially near the nanowire 330 and the speed of the nucleic acid chain 100. If other molecule sensors 300 are configured in the nanochannel 240, sensitive to the other bases 120 (or complimentary oligonucleotides 124), a complete solution of the nucleic acid chain 100 may be derived based on the velocity of the nucleic acid chain 100, the relative positioning of the various molecule sensors 300, and the corresponding conductance change in the various molecule sensors 300.

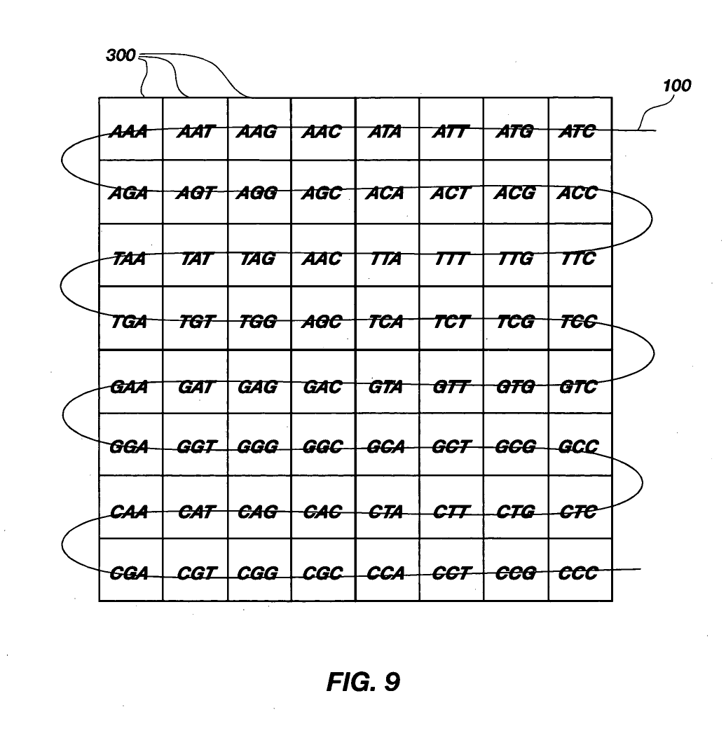

FIG. 9 illustrates a molecule analysis device with a plurality of molecule sensors 300 coupled by a long nanochannel (not shown) for carrying the nucleic acid chain 100. The embodiment of FIG. 9 may be used to detect a variety of different sequences of interest in the nucleic acid chain 100. For example, the embodiment illustrated in FIG. 9 is configured to detect all the 64 possible combinations of codons. This may be useful for identifying the various amino acids, start codons, and stop codons of a protein. It will be clear to those of ordinary skill in the art that many other useful combinations of molecule analysis devices with various combinations of oligonucleotides 124, nitrogenous material 350, or combinations thereof are contemplated with the scope of the present invention.

Of course, it will also be clear that the matrix organization is an arbitrary organization useful for explanation and illustration. However, may other configurations including straight nanochannels, curved nanochannels, serpentine nanochannels, and various organizations of the molecule sensors are contemplated within the scope of the present invention.

Although the foregoing description contains many specifics, these are not to be construed as limiting the scope of the present invention, but merely as providing certain exemplary embodiments. Similarly, other embodiments of the invention may be devised which do not depart from the spirit or scope of the present invention. The scope of the invention is, therefore, indicated and limited only by the appended claims and their legal equivalents, rather than by the foregoing description. All additions, deletions, and modifications to the invention, as disclosed herein, which fall within the meaning and scope of the claims, are encompassed by the present invention.

What is claimed is:

1. A molecular analysis device, comprising:
a molecule sensor comprising;
a nanowire operably coupling a first terminal and a second terminal;
a nitrogenous material disposed on the nanowire, the nitrogenous material configured to interact with an identifiable configuration of a molecule; and
wherein the molecule sensor develops a conductance change responsive to the interaction; and
a molecule guide configured for guiding at least a portion of the molecule near the molecule sensor to enable the interaction.

2. The device of claim 1, wherein the interaction comprises a transitory chemical bond between the nitrogenous material and the at least a portion of the molecule substantially near the molecule sensor.

3. The device of claim 1, wherein the identifiable configuration comprises a base selected from the group consisting of adenine, thymine, uracil, cytosine, and guanine.

4. The device of claim 1, wherein the nitrogenous material comprises a base selected from the group consisting of adenine, thymine, uracil, cytosine, and guanine.

5. The device of claim 4, wherein the nitrogenous material further comprises a material selected from the group consisting of a sugar chemically bonded to the base and a sugar-phosphate chemically bonded to the base.

6. The device of claim 1, wherein the nitrogenous material comprises an oligonucleotide and the identifiable configuration of the molecule is a complementary match to the oligonucleotide.

7. The device of claim 1, wherein the nanowire includes a p-type doping and the conductance change comprises a measurable increase in conductance.

8. The device of claim 1, wherein the nanowire includes an n-type doping and the conductance change comprises a measurable decrease in conductance.

9. The device of claim 1, wherein the molecule guide comprises:
a nanochannel including an entrance point and an exit point, the nanochannel configured for substantially straightening the molecule and guiding the molecule near the nitrogenous material; and
a transport medium disposed in the nanochannel and configured for transporting the molecule in a lengthwise fashion through the nanochannel in a direction from the entrance point to the exit point to successively present each segment of a plurality of segments distributed along the length of the molecule to the nitrogenous material.

10. The device of claim 9, wherein the transport medium near the exit point is positively charged relative to the transport medium near the entrance point to cause the molecule to transport in the transport direction.

11. The device of claim 9, wherein the nanowire is positioned at a location selected from the group consisting of: substantially in the nanochannel between the entrance point and the exit point; external to the nanochannel and substantially near the entrance point of the nanochannel; and external to the nanochannel and substantially near the exit point of the nanochannel.

12. The device of claim 1, wherein the molecule guide comprises:
a nanopore formed in a membrane, the nanopore including an entrance point and an exit point and configured for guiding the molecule substantially near the nitrogenous material; and
a transport medium configured for transporting the molecule through the nanopore in a transport direction from the entrance point to the exit point to successively present each segment of a plurality of segments distributed along the length of the molecule to the nitrogenous material.

13. The device of claim 12, wherein the transport medium near the exit point is positively charged relative to the transport medium near the entrance point.

14. The device of claim 12, wherein the nanowire is positioned at a location near the entrance point of the nanopore or near the exit point of the nanopore.

15. A molecular analysis device, comprising:
a plurality of molecule sensors, each molecule sensor of the plurality comprising:
a nanowire operably coupling a first terminal and a second terminal;
a nitrogenous material disposed on the nanowire and configured to interact with an identifiable configuration of a molecule; and
wherein the molecule sensor develops a conductance change responsive to the interaction; and
a molecule guide configured for guiding at least a portion of the molecule near the nitrogenous material of each molecule sensor of the plurality to enable the interaction.

16. The device of claim 15, wherein the nanowire of each molecule sensor of the plurality is configured with one of a plurality of nitrogenous materials disposed thereon, and wherein each nitrogenous material of the plurality is configured to interact with a different identifiable configuration of the molecule.

17. The device of claim 15, wherein the plurality of molecule sensors are configured to detect a plurality of identifiable configurations.

18. The device of claim 15, wherein the chemical reaction of each molecule sensor of the plurality comprises a transitory chemical bond between the nitrogenous material and the at least a portion of the molecule substantially near each molecule sensor of the plurality.

19. The device of claim 15, wherein the identifiable configuration comprises a base selected from the group consisting of adenine, thymine, uracil, cytosine, and guanine.

20. The device of claim 15, wherein the nitrogenous material disposed on the nanowire of each molecule sensor of the plurality comprises a base selected from the group consisting of adenine, thymine, uracil, cytosine, and guanine.

21. The device of claim 20, wherein the nitrogenous material further comprises a material selected from the group consisting of a sugar chemically bonded to the base and a sugar-phosphate chemically bonded to the base.

22. The device of claim 15, wherein the nitrogenous material disposed on the nanowire of each molecule sensor of the plurality comprises an oligonucleotide and the identifiable configuration of the molecule is a complementary match to the oligonucleotide.

23. The device of claim 15, wherein the nanowire of at least one molecule sensor of the plurality includes a p-type doping and the conductance change comprises a measurable increase in conductance.

24. The device of claim 15, wherein the nanowire of at least one molecule sensor of the plurality includes an n-type doping and the conductance change comprises a measurable decrease in conductance.

25. The device of claim 15, wherein the molecule guide comprises:
a nanochannel including an entrance point and an exit point, the nanochannel configured for substantially straightening the molecule and guiding the molecule substantially near the nitrogenous material of each molecule sensor of the plurality; and
a transport medium disposed in the nanochannel and configured for transporting the molecule in a lengthwise fashion through the nanochannel in a direction from the entrance point to the exit point to successively present each segment of a plurality of segments distributed along the length of the molecule to the nitrogenous material of each molecule sensor of the plurality.

26. The device of claim 25, wherein the transport medium near the exit point is positively charged relative to the transport medium near the entrance point.

27. The device of claim 25, wherein the nanowire of each molecule sensor of the plurality is positioned at a location selected from the group consisting of:
substantially in the nanochannel between the entrance point and the exit point; external to the nanochannel and substantially near the entrance point of the nanochannel; and external to the nanochannel and substantially near the exit point of the nanochannel.

28. The device of claim 15, wherein the molecule guide comprises:
a nanopore formed in a membrane, the nanopore including an entrance point and an exit point and configured for guiding the molecule near the nitrogenous material of each molecule sensor of the plurality; and
a transport medium configured for transporting the molecule through the nanopore in a direction from the entrance point to the exit point to successively present each segment of a plurality of segments distributed along the length of the molecule to the nitrogenous material of each molecule sensor of the plurality.

29. The device of claim 28, wherein the transport medium near the exit point is positively charged relative to the transport medium near the entrance point.

30. The device of claim 28, wherein the nanowire of each molecule sensor of the plurality is positioned at a location selected from the group consisting of near the entrance point of the nanopore and near the exit point of the nanopore.

31. A method of detecting a molecule, comprising:
guiding at least a portion of the molecule substantially near a molecule sensor, the molecule sensor including a nanowire disposed in a molecule guide;
interacting an identifiable configuration of the molecule and a nitrogenous material disposed on the nanowire; and
sensing a conductance change in the molecule sensor responsive to the interaction.

32. The method of claim 31, further comprising:
guiding at least one additional portion of the molecule substantially near at least one additional molecule sensor, the at least one additional molecule sensor including at least one additional nanowire;
developing at least one additional chemical reaction between at least one additional identifiable configuration of the molecule and at least one additional nitrogenous material disposed on the at least one additional nanowire of the at least one additional molecule sensor; and
sensing at least one additional conductance change in the at least one additional molecule sensor responsive to the at least one additional chemical reaction.

33. The method of claim 31, wherein developing the chemical reaction comprises producing a transitory chemical bond between the nitrogenous material and the at least a portion of the molecule near the molecule sensor.

34. The method of claim 31, wherein the identifiable configuration of the molecule comprises a base selected from the group consisting of adenine, thymine, uracil, cytosine, and guanine.

35. The method of claim 31, wherein the nitrogenous material comprises a base selected from the group consisting of adenine, thymine, uracil, cytosine, and guanine.

36. The method of claim 35, wherein the nitrogenous material further comprises a material selected from the group consisting of a sugar chemically bonded to the base and a sugar-phosphate chemically bonded to the base.

37. The method of claim 31, wherein the nitrogenous material comprises an oligonucleotide and the identifiable configuration of the molecule is a complementary match to the oligonucleotide.

38. The method of claim 31, wherein sensing the conductance change further comprises sensing a measurable increase in conductance of the nanowire, wherein the nanowire includes a p-type doping.

39. The method of claim 31, wherein sensing the conductance change further comprises sensing a measurable decrease in conductance of the nanowire, wherein the nanowire includes an n-type doping.

40. The method of claim 31, wherein guiding at least a portion of the molecule further comprises transporting the molecule in a transport medium in a lengthwise fashion through a nanochannel to successively present each segment of a plurality of segments distributed along the length of the molecule to the nitrogenous material.

41. The method of claim 40, wherein transporting the molecule further comprises applying a more positive charge to the transport medium near an exit point of the nanochannel relative to a charge of the transport medium near an entrance point of the nanochannel.

42. The method of claim 31, wherein guiding at least a portion of the molecule further comprises transporting the molecule in a transport medium in a lengthwise fashion through a nanopore formed in a membrane to successively present each segment of a plurality of segments distributed along the length of the molecule to the nitrogenous material.

43. The method of claim 42, wherein transporting the molecule further comprises applying a more positive charge to the transport medium near an exit point of the nanopore relative to a charge of the transport medium near an entrance point of the nanopore.
